(19) United States
(12) Patent Application Publication
(10) Pub. No.: US 2026/0306390 A1
(43) Pub. Date: Oct. 8, 2026

(54) PERSONAL CARE RINSE OFF FORMULATION

(71) Applicants: Dow Global Technologies LLC, Midland, MI (US); Rohm and Haas Company, Collegeville, PA (US); Dow Silicones Corporation, Midland, MI (US)

(72) Inventors: Janet McMillan, Bay City, MI (US); Daniel S. Miller, Phoenixville, PA (US); Robert D. Kennedy, Midland, MI (US); Matthew M. Yonkey, Sanford, MI (US); Lauren Wilson, Midland, MI (US); Tanner McDaniel, Midland, MI (US); Timothy J. Young, Bay City, MI (US); Bethany K. Johnson, Midland, MI (US)

(21) Appl. No.: 19/482,024

(22) PCT Filed: May 23, 2024
(86) PCT No.: PCT/US2024/030677

§ 371 Date: Nov. 6, 2025

Related U.S. Application Data
(60) Provisional application No. 63/470,461, filed on Jun. 2, 2023.

(51) Int. Cl. A61K 8/73; A61K 8/20; A61K 8/44; A61K 8/46; A61Q 5/02; A61Q 19/10

(52) U.S. Cl. CPC A61K 8/73; A61K 8/20; A61K 8/44; A61K 8/463; A61Q 5/02; A61Q 19/10; A61K 2800/48; A61K 2800/596; A61K 2800/805

(57) ABSTRACT

An aqueous personal care rinse off formulation is provided, comprising: a dermatologically acceptable aqueous vehicle; a dermatologically acceptable cleaning surfactant, wherein the dermatologically acceptable cleaning surfactant comprises an anionic surfactant; and an alkyl polysaccharide thickener; wherein the alkyl polysaccharide thickener comprises 50 to 100 wt %, based on weight of the alkyl polysaccharide thickener, of a linear alkyl polysaccharide of formula A R—O—(S)nH (A) wherein R is a linear C₉₋₁₅ alkyl group; wherein n is an average of 1 to 2.5 and wherein S is a monosaccharide selected from the group consisting of ribulose, xylulose, ribose, arabinose, xylose, lyxose and mixtures thereof; and wherein 70 to 100 wt % of the monosaccharide S, based on weight of monosaccharide S in the linear alkyl polysaccharide of formula A, is xylose.

The present invention relates to an aqueous personal care rinse off formulation. In particular, the present invention relates to an aqueous personal care rinse off formulation, comprising: a dermatologically acceptable aqueous vehicle; a dermatologically acceptable cleaning surfactant, wherein the dermatologically acceptable cleaning surfactant comprises an anionic surfactant; and an alkyl polysaccharide thickener, wherein the alkyl polysaccharide thickener comprises 50 to 100 wt %, based on weight of the alkyl polysaccharide thickener, of a linear alkyl polysaccharide of formula A

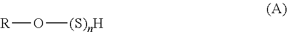

wherein R is a linear C₉₋₁₅ alkyl group; wherein n is an average of 1 to 2.5 and wherein S is a monosaccharide selected from the group consisting of ribulose, xylulose, ribose, arabinose, xylose, lyxose and mixtures thereof; and wherein 70 to 100 wt % of the monosaccharide S, based on weight of monosaccharide S in the linear alkyl polysaccharide of formula A, is xylose; with the proviso that the alkyl polysaccharide thickener comprises <15 wt %, based on weight of the alkyl polysaccharide thickener, of branched alkyl polysaccharide of formula I

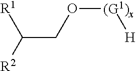

wherein R¹ is a linear or branched C₃₋₄ alkyl group; wherein R² is a linear or branched C₅₋₆ alkyl group; wherein G¹ is a monosaccharide with 4 to 6 carbon atoms and wherein x is 1.1 to 4; with the proviso that the alkyl polysaccharide thickener comprises <1.5 wt %, based on weight of the alkyl polysaccharide thickener, of branched alkylpolysaccharide of formula II

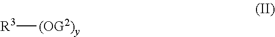

wherein R³ is an alkyl or alkenyl moiety containing 7 to 30 carbon atoms with branching at the 2 or higher carbon position on R³, wherein the branching is selected from the group consisting of methyl, ethyl, isopropyl, n-propyl, butyl, pentyl, hexyl and mixtures thereof; wherein G² is a saccharide moiety selected from the group consisting of glucose, fructose, mannose, galactose, talose, gulose, allose, altrose, idose, arabinose, xylose, lyxose, ribose and mixtures thereof and wherein y is an average of ≥2.

Surfactant based formulations used in rinse off personal care applications, which are most commonly formulated using sulfate based anionic surfactants in mixtures with amphoteric surfactants are typically thickened through the addition of sodium chloride, which induces a spherical to worm like micelle transition in the microstructure of the surfactants resulting in the desired viscosity. However, thickening through the addition of salt alone can pose problems, including undesired interactions and reduction in performance with deposition aid polymers; reduction in stability of emulsions used in personal care formulations, limited efficacy for thickening higher ethoxylate sulfate surfactants (e.g., sodium laureth sulfate with an average degree of ethoxylation of three (3EO SLES)).

To solve these problems with salt thickening, polymer rheology modifiers have been used alone, or in combination with salts, to provide desired thickening to personal care formulations. However, conventional polymer rheology modifiers tend to exhibit poor biodegradability, are typically derived from petroleum and as such are not considered to be environmentally acceptable or renewable materials.

Alkyl polyglucosides have been proposed as an alternative for use as thickeners in mixed surfactant personal care systems. However, such alkyl polyglucosides show limited efficacy for thickening sulfate based surfactant systems to desirable viscosity with low use levels.

Accordingly, there remains a need for alternative thickeners that are effective at thickening a variety of sulfate based personal care formulations, but also are naturally derived and biodegradable with an improved sustainability profile.

The present invention provides an aqueous personal care rinse off formulation comprising: a dermatologically acceptable aqueous vehicle; a dermatologically acceptable cleaning surfactant, wherein the dermatologically acceptable cleaning surfactant comprises an anionic surfactant; and an alkyl polysaccharide thickener, wherein the alkyl polysaccharide thickener comprises 50 to 100 wt %, based on weight of the alkyl polysaccharide thickener, of a linear alkyl polysaccharide of formula A

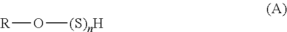

wherein R is a linear C₉₋₁₅ alkyl group; wherein n is an average of 1 to 2.5 and wherein S is a monosaccharide selected from the group consisting of ribulose, xylulose, ribose, arabinose, xylose, lyxose and mixtures thereof; and wherein 70 to 100 wt % of the monosaccharide S, based on weight of monosaccharide S in the linear alkyl polysaccharide of formula A, is xylose; with the proviso that the alkyl polysaccharide thickener comprises <15 wt %, based on weight of the alkyl polysaccharide thickener, of branched alkyl polysaccharide of formula I

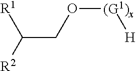

wherein R¹ is a linear or branched C₃₋₄ alkyl group; wherein R² is a linear or branched C₅₋₆ alkyl group; wherein G¹ is a monosaccharide with 4 to 6 carbon atoms and wherein x is 1.1 to 4; and with the proviso that the alkyl polysaccharide thickener comprises <1.5 wt %, based on weight of the alkyl polysaccharide thickener, of branched alkylpolysaccharide of formula II

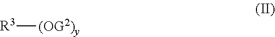

wherein R³ is an alkyl or alkenyl moiety containing 7 to 30 carbon atoms with branching at the 2 or higher carbon position on R³, wherein the branching is selected from the group consisting of methyl, ethyl, isopropyl, n-propyl, butyl, pentyl, hexyl and mixtures thereof; wherein G² is a saccharide moiety selected from the group consisting of glucose, fructose, mannose, galactose, talose, gulose, allose, altrose, idose, arabinose, xylose, lyxose, ribose and mixtures thereof; and wherein y is an average of ≥2.

The present invention provides an aqueous personal care rinse off formulation comprising: a dermatologically acceptable aqueous vehicle; a dermatologically acceptable cleaning surfactant, wherein the dermatologically acceptable cleaning surfactant comprises an anionic surfactant; and an alkyl polysaccharide thickener, wherein the alkyl polysaccharide thickener comprises 50 to 100 wt %, based on weight of the alkyl polysaccharide thickener, of a linear alkyl polysaccharide of formula A

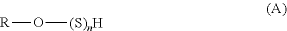

wherein R is a linear C₉₋₁₅ alkyl group; wherein n is an average of 1 to 2.5 and wherein S is a monosaccharide selected from the group consisting of ribulose, xylulose, ribose, arabinose, xylose, lyxose and mixtures thereof; and wherein 70 to 100 wt % of the monosaccharide S, based on weight of monosaccharide S in the linear alkyl polysaccharide of formula A, is xylose; with the proviso that the alkyl polysaccharide thickener comprises <15 wt %, based on weight of the alkyl polysaccharide thickener, of branched alkyl polysaccharide of formula I

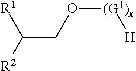

wherein R¹ is a linear or branched C₃₋₄ alkyl group; wherein R² is a linear or branched C₅₋₆ alkyl group; wherein G¹ is a monosaccharide with 4 to 6 carbon atoms and wherein x is 1.1 to 4; with the proviso that the alkyl polysaccharide thickener comprises <1.5 wt %, based on weight of the alkyl polysaccharide thickener, of branched alkylpolysaccharide of formula II

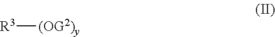

wherein R³ is an alkyl or alkenyl moiety containing 7 to 30 carbon atoms with branching at the 2 or higher carbon position on R³, wherein the branching is selected from the group consisting of methyl, ethyl, isopropyl, n-propyl, butyl, pentyl, hexyl and mixtures thereof; wherein G² is a saccharide moiety selected from the group consisting of glucose, fructose, mannose, galactose, talose, gulose, allose, altrose, idose, arabinose, xylose, lyxose, ribose and mixtures thereof and wherein y is an average of ≥2; and wherein the aqueous personal care rinse off formulation has a viscosity ≥2,000 cP at 21° C.

The present invention provides a method of cleaning at least one of mammalian skin and hair, comprising: applying an aqueous personal care rinse off formulation of the present invention to the skin or hair of a mammal; and rinsing the aqueous personal care rinse off formulation from the skin or hair with a rinse water.

### DETAILED DESCRIPTION

We have surprisingly found that aqueous personal care rinse off formulations comprising sulfate based surfactants may be effectively thickened using an alkyl polysaccharide thickener; wherein the alkyl polysaccharide thickener comprises 50 to 100 wt %, based on weight of the alkyl polysaccharide thickener, of a linear alkyl polysaccharide of formula A

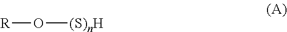

wherein R is a linear C₉₋₁₅ alkyl group; wherein n is an average of 1 to 2.5 and wherein S is a monosaccharide selected from the group consisting of ribulose, xylulose, ribose, arabinose, xylose, lyxose and mixtures thereof; and wherein 70 to 100 wt % of the monosaccharide S, based on weight of monosaccharide S in the linear alkyl polysaccharide of formula A, is xylose; and that the linear alkyl polysaccharide of formula A of the present invention provide dramatically improved thickening performance over linear alkyl polyglucosides in such formulations.

Unless otherwise indicated, ratios, percentages, parts, and the like are by weight.

As used herein, unless otherwise indicated, the phrase “molecular weight” or MW refers to the weight average molecular weight as measured in a conventional manner with gel permeation chromatography (GPC) and conventional standards, such as polystyrene molecular weight standards. GPC techniques are discussed in detail in Modern Size Exclusion Liquid Chromatography—Practice of Gel Permeation and Gel Filtration Chromatography, Second Edition, A. M. Striegel, W. W. Yau, J. J. Kirkland, D. D. Bly; John Wiley & Sons, Inc. 2009. Molecular weights are reported herein in units of Daltons, or equivalently, g/mol.

The term “dermatologically acceptable” as used herein and in the appended refers to ingredients that are typically used for topical application to the skin, and is intended to underscore that materials that are toxic when present in the amounts typically found in skin care compositions are not contemplated as part of the present invention.

Preferably, the aqueous personal care rinse off formulation of the present invention is selected from the group consisting of a shampoo, a conditioning shampoo, a body wash, an exfoliating body wash, a facial wash, an exfoliating facial wash and a liquid hand soap. More preferably, the aqueous personal care rinse off formulation of the present invention is selected from the group consisting of a shampoo, a conditioning shampoo, body wash, a facial wash and a liquid hand soap. Most preferably, the aqueous personal care rinse off formulation of the present invention is selected from the group consisting of a shampoo and a conditioning shampoo.

Preferably, the aqueous personal care rinse off formulation of the present invention, comprises: a dermatologically acceptable aqueous vehicle (preferably, wherein the aqueous personal care rinse off formulation comprises 25 to 98.9 wt % (preferably, 30 to 97.5 wt %; more preferably, 40 to 96.4 wt %; most preferably, 50 to 95 wt %), based on weight of the aqueous personal care rinse off formulation, of a dermatologically acceptable aqueous vehicle); a dermatologically acceptable cleaning surfactant (preferably, wherein the aqueous personal care rinse off formulation comprises 1 to 50 wt % (preferably, 2 to 40 wt %; more preferably, 3 to 30 wt %; most preferably, 4 to 20 wt %), based on weight of the aqueous personal care rinse off formulation, of a dermatologically acceptable cleaning surfactant), wherein the dermatologically acceptable cleaning surfactant comprises an anionic surfactant; and an alkyl polysaccharide thickener (preferably, wherein the aqueous personal care rinse off formulation comprises 0.1 to 30 wt % (preferably, 0.5 to 25 wt %; more preferably, 0.6 to 20 wt %; most preferably, 1 to 15 wt %), based on weight of the aqueous personal care rinse off formulation, of an alkyl polysaccharide thickener), wherein the alkyl polysaccharide thickener comprises 50 to 100 wt % (preferably, 75 to 100 wt %; more preferably, 80 to 100 wt %; still more preferably, 90 to 100 wt %; still more preferably, 95 to 100 wt %; still yet more preferably, 98 to 100 wt %; yet still more preferably, 99 to 100 wt %; most preferably, 100 wt %), based on weight of the alkyl polysaccharide thickener, of a linear alkyl polysaccharide of formula A

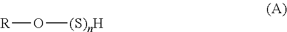

wherein R is a linear C₉₋₁₅ alkyl group (preferably, a C₉₋₁₄ alkyl group; more preferably, a C₉₋₁₃ alkyl group; most preferably, a C₁₀₋₁₂ alkyl group); wherein n is an average of 1 to 2.5 (preferably, 1.1 to 2; more preferably, 1.1 to 1.75; most preferably, 1.15 to 1.5) and wherein S is a monosaccharide selected from the group consisting of ribulose, xylulose, ribose, arabinose, xylose, lyxose and mixtures thereof (preferably, arabinose, xylose and mixtures thereof; most preferably, xylose); and wherein 70 to 100 wt % of the monosaccharide S (preferably, 80 to 100 wt %; more preferably, 85 to 100 wt %; still more preferably, 90 to 100 wt %; yet more preferably, 95 to 100 wt %; still yet more preferably, 98 to 100 wt %; most preferably, 99 to 100 wt %), based on weight of monosaccharide S in the linear alkyl polysaccharide of formula A, is xylose; with the proviso that the alkyl polysaccharide thickener comprises <15 wt % (preferably, <10 wt %; more preferably. <1 wt %; still more preferably, <0.1 wt %; yet more preferably, 0.01 wt %; still yet more preferably, 0.001 wt %; most preferably, less than detectable limit), based on weight of the alkyl polysaccharide thickener, of branched alkyl polysaccharide of formula I

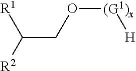

wherein R¹ is a linear or branched C₃₋₄ alkyl group; wherein R² is a linear or branched C₅₋₆ alkyl group; wherein G¹ is a monosaccharide with 4 to 6 carbon atoms and wherein x is 1.1 to 4; and with the proviso that the alkyl polysaccharide thickener comprises <1.5 wt % (preferably, <1 wt %; more preferably, <0.5 wt %; still more preferably, <0.1 wt %; yet more preferably, <0.01 wt %; still yet more preferably, <0.001 wt %; yet still more preferably, <0.0001 wt %; most preferably, less than detectable limit), based on weight of the alkyl polysaccharide thickener, of branched alkylpolysaccharide of formula II

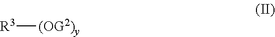

wherein R³ is an alkyl or alkenyl moiety containing 7 to 30 carbon atoms with branching at the 2 or higher carbon position on R³, wherein the branching is selected from the group consisting of methyl, ethyl, isopropyl, n-propyl, butyl, pentyl, hexyl and mixtures thereof; wherein G² is a saccharide moiety selected from the group consisting of glucose, fructose, mannose, galactose, talose, gulose, allose, altrose, idose, arabinose, xylose, lyxose, ribose and mixtures thereof and wherein y is an average of ≥2 (preferably, 2 to 10).

More preferably, the aqueous personal care rinse off formulation of the present invention, comprises: a dermatologically acceptable aqueous vehicle (preferably, wherein the aqueous personal care rinse off formulation comprises 25 to 98.9 wt % (preferably, 30 to 97.5 wt %; more preferably, 40 to 96.4 wt %; most preferably, 50 to 95 wt %), based on weight of the aqueous personal care rinse off formulation, of a dermatologically acceptable aqueous vehicle); a dermatologically acceptable cleaning surfactant (preferably, wherein the aqueous personal care rinse off formulation comprises 1 to 50 wt % (preferably, 2 to 40 wt %; more preferably, 3 to 30 wt %; most preferably, 4 to 20 wt %), based on weight of the aqueous personal care rinse off formulation, of a dermatologically acceptable cleaning surfactant), wherein the dermatologically acceptable cleaning surfactant comprises an anionic surfactant; and an alkyl polysaccharide thickener (preferably, wherein the aqueous personal care rinse off formulation comprises 0.1 to 30 wt % (preferably, 0.5 to 25 wt %; more preferably, 0.6 to 20 wt %; most preferably, 1 to 15 wt %), based on weight of the aqueous personal care rinse off formulation, of an alkyl polysaccharide thickener), wherein the alkyl polysaccharide thickener comprises 50 to 100 wt % (preferably, 75 to 100 wt %; more preferably, 80 to 100 wt %; still more preferably, 90 to 100 wt %; still more preferably, 95 to 100 wt %; still yet more preferably, 98 to 100 wt %; yet still more preferably, 99 to 100 wt %; most preferably, 100 wt %), based on weight of the alkyl polysaccharide thickener, of a linear alkyl polysaccharide of formula A

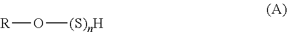

wherein R is a linear C₉₋₁₅ alkyl group (preferably, a C₉₋₁₄ alkyl group; more preferably, a C₉₋₁₃ alkyl group; most preferably, a C₁₀₋₁₂ alkyl group); wherein n is an average of 1 to 2.5 (preferably, 1.1 to 2; more preferably, 1.1 to 1.75; most preferably, 1.15 to 1.5) and wherein S is a monosaccharide selected from the group consisting of ribulose, xylulose, ribose, arabinose, xylose, lyxose and mixtures thereof (preferably, arabinose, xylose and mixtures thereof; most preferably, xylose); and wherein 70 to 100 wt % of the monosaccharide S (preferably, 80 to 100 wt %; more preferably, 85 to 100 wt %; still more preferably, 90 to 100 wt %; yet more preferably, 95 to 100 wt %; still yet more preferably, 98 to 100 wt %; most preferably, 99 to 100 wt %), based on weight of monosaccharide S in the linear alkyl polysaccharide of formula A, is xylose; wherein the linear alkyl polysaccharide of formula A comprises 0.1 to 39 wt % (preferably, 1 to 25 wt %) of a combined weight of the linear alkyl polysaccharide of formula A and the dermatologically acceptable cleaning surfactant in the aqueous personal care rinse off composition; with the proviso that the alkyl polysaccharide thickener comprises <15 wt % (preferably, <10 wt %; more preferably, <1 wt %; still more preferably, <0.1 wt %; yet more preferably, 0.01 wt %; still yet more preferably, 0.001 wt %; most preferably, less than detectable limit), based on weight of the alkyl polysaccharide thickener, of branched alkyl polysaccharide of formula I

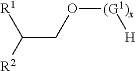

wherein R¹ is a linear or branched C₃₋₄ alkyl group; wherein R² is a linear or branched C₅₋₆ alkyl group; wherein G¹ is a monosaccharide with 4 to 6 carbon atoms and wherein x is 1.1 to 4; and with the proviso that the alkyl polysaccharide thickener comprises <1.5 wt % (preferably, <1 wt %; more preferably, <0.5 wt %; still more preferably, <0.1 wt %; yet more preferably, <0.01 wt %; still yet more preferably, <0.001 wt %; yet still more preferably, <0.0001 wt %; most preferably, less than detectable limit), based on weight of the alkyl polysaccharide thickener, of branched alkylpolysaccharide of formula II

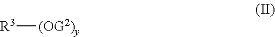

wherein R³ is an alkyl or alkenyl moiety containing 7 to 30 carbon atoms with branching at the 2 or higher carbon position on R³, wherein the branching is selected from the group consisting of methyl, ethyl, isopropyl, n-propyl, butyl, pentyl, hexyl and mixtures thereof; wherein G² is a saccharide moiety selected from the group consisting of glucose, fructose, mannose, galactose, talose, gulose, allose, altrose, idose, arabinose, xylose, lyxose, ribose and mixtures thereof and wherein y is an average of ≥2 (preferably, 2 to 10).

Preferably, the aqueous personal care rinse off formulation of the present invention, comprises a dermatologically acceptable aqueous vehicle. More preferably, the aqueous personal care rinse off formulation of the present invention, comprises: 25 to 98.9 wt % (preferably, 30 to 97.5 wt %; more preferably, 40 to 96.4 wt %; most preferably, 50 to 95 wt %), based on weight of the aqueous personal care rinse off formulation, of a dermatologically acceptable aqueous vehicle. Still more preferably, the aqueous personal care rinse off formulation of the present invention, comprises: 25 to 98.9 wt % (preferably, 30 to 97.5 wt %; more preferably, 40 to 96.4 wt %; most preferably, 50 to 95 wt %), based on weight of the aqueous personal care rinse off formulation, of a dermatologically acceptable aqueous vehicle; wherein the dermatologically acceptable aqueous vehicle comprises water. Yet more preferably, the aqueous personal care rinse off formulation of the present invention, comprises: 25 to 98.9 wt % (preferably, 30 to 97.5 wt %; more preferably, 40 to 96.4 wt %; most preferably, 50 to 95 wt %), based on weight of the aqueous personal care rinse off formulation, of a dermatologically acceptable aqueous vehicle; wherein the dermatologically acceptable aqueous vehicle is selected from the group consisting of water and an aqueous C-A alcohol mixture. Most preferably, the aqueous personal care rinse off formulation of the present invention, comprises: 25 to 98.9 wt % (preferably, 30 to 97.5 wt %; more preferably, 40 to 96.4 wt %; most preferably, 50 to 95 wt %), based on weight of the aqueous personal care rinse off formulation, of a dermatologically acceptable aqueous vehicle, wherein the dermatologically acceptable aqueous vehicle is water.

Preferably, the water used in the aqueous personal care rinse off formulation of the present invention is at least one of distilled water and deionized water. More preferably, the water used in the aqueous personal care rinse off formulation of the present invention is distilled and deionized.

Preferably, the aqueous personal care rinse off formulation of the present invention comprises 1 to 50 wt % (preferably, 2 to 40 wt %; more preferably, 3 to 30 wt %; most preferably, 4 to 20 wt %), based on weight of the aqueous personal care rinse off formulation, of a dermatologically acceptable cleaning surfactant; wherein the dermatologically acceptable cleaning surfactant comprises an anionic surfactant. More preferably, the aqueous personal care rinse off formulation of the present invention comprises 1 to 50 wt % (preferably, 2 to 40 wt %; more preferably, 3 to 30 wt %; most preferably, 4 to 20 wt %), based on weight of the aqueous personal care rinse off formulation, of a dermatologically acceptable cleaning surfactant; wherein the dermatologically acceptable cleaning surfactant comprises an anionic surfactant selected from the group consisting of glycinates (e.g., sodium cocoyl glycinate), taurates (e.g., sodium methyl cocoyl taurate), glutamates (e.g., sodium cocoyl glutamate), sarcosinates (e.g., sodium lauroyl sarcosinate), isethionates (e.g., sodium cocoyl isethionate, sodium lauroyl methyl isethionate), sulfoacetates (e.g., sodium lauryl sulfoacetate), alaninates (e.g., sodium cocoyl alaninate), sulfates (e.g., sodium laureth sulfate (SLES), sodium lauryl sulfate (SLS)), sulfonates (e.g., sodium C₁₄₋₁₆ olefin sulfonate), succinates (e.g., disodium lauryl sulfosuccinate), and mixtures thereof. Most preferably, the aqueous personal care rinse off formulation of the present invention comprises 1 to 50 wt % (preferably, 2 to 40 wt %; more preferably, 3 to 30 wt %; most preferably, 4 to 20 wt %), based on weight of the aqueous personal care rinse off formulation, of a dermatologically acceptable cleaning surfactant; wherein the dermatologically acceptable cleaning surfactant comprises an anionic surfactant including sodium laureth sulfate (SLES).

Preferably, the aqueous personal care rinse off formulation of the present invention comprises 1 to 50 wt % (preferably, 2 to 40 wt %; more preferably, 3 to 30 wt %; most preferably, 4 to 20 wt %), based on weight of the aqueous personal care rinse off formulation, of a dermatologically acceptable cleaning surfactant; wherein the dermatologically acceptable cleaning surfactant comprises a mixture of an anionic surfactant and a amphoteric surfactant. More preferably, the aqueous personal care rinse off formulation of the present invention comprises 1 to 50 wt % (preferably, 2 to 40 wt %; more preferably, 3 to 30 wt %; most preferably, 4 to 20 wt %), based on weight of the aqueous personal care rinse off formulation, of a dermatologically acceptable cleaning surfactant; wherein the dermatologically acceptable cleaning surfactant comprises a mixture of an anionic surfactant and a amphoteric surfactant; wherein the anionic surfactant is selected from the group consisting of glycinates (e.g., sodium cocoyl glycinate), taurates (e.g., sodium methyl cocoyl taurate), glutamates (e.g., sodium cocoyl glutamate), sarcosinates (e.g., sodium lauroyl sarcosinate), isethionates (e.g., sodium cocoyl isethionate, sodium lauroyl methyl isethionate), sulfoacetates (e.g., sodium lauryl sulfoacetate), alaninates (e.g., sodium cocoyl alaninate), sulfates (e.g., sodium laureth sulfate (SLES)), sulfonates (e.g., sodium C₁₄₋₁₆ olefin sulfonate), succinates (e.g., disodium lauryl sulfosuccinate), carboxylates (e.g., sodium lauryl glucose carboxylate) and mixtures thereof; and wherein the amphoteric surfactant is selected from the group consisting of betaines (e.g., alkyl betaines such as trimethylglycine and cetyl betaine; and amido betaines such as cocamidopropyl betaine); amphoacetates (e.g., sodium cocoamphoacetate); sultaines (e.g., cocamidopropyl hydroxysultaine) and mixtures thereof. Most preferably, the aqueous personal care rinse off formulation of the present invention comprises 1 to 50 wt % (preferably, 2 to 40 wt %; more preferably, 3 to 30 wt %; most preferably, 4 to 20 wt %), based on weight of the aqueous personal care rinse off formulation, of a dermatologically acceptable cleaning surfactant; wherein the dermatologically acceptable cleaning surfactant comprises a mixture of sodium laureth sulfate (SLES) and cocamidopropyl betaine.

The dermatologically acceptable cleaning surfactant optionally further comprises a cationic surfactant. Cationic surfactants include quaternary ammonia compounds (e.g., behentrimonium chloride, cetrimonium chloride) or amine-based surfactants (e.g., stearamidopropyl dimethylamine).

The dermatologically acceptable cleaning surfactant optionally further comprises a nonionic surfactant with the proviso that when the dermatologically acceptable cleaning surfactant comprises a nonionic surfactant other than the alkyl polysaccharide thickener, the weight ratio of the anionic surfactant to the nonionic surfactant in the aqueous personal care rinse off formulation is greater than 1:1 (preferably, >2:1; more preferably >2.5:1) (i.e., the dermatologically acceptable anionic cleaning surfactant predominates over any nonionic surfactant present, excluding the alkyl polysaccharide thickener) (i.e., the alkyl polysaccharide thickener is not considered a nonionic surfactant for purposes of the anionic to nonionic cleaning surfactant weight ratio). Nonionic surfactants include fatty alcohols (e.g., cetearyl alcohol, stearyl alcohol, cetyl alcohol, lauryl alcohol) and fatty alkanolamides (e.g., cocamide monoethanolamine, cocamide, diethanolamine, soyamide diethanolamine, lauramide diethanolamine, oleamide monoisopropanolamine, stearamide monoethanolamine, myristamide monoethanolamine, lauramide monoethanolamine, capramide diethanolamine, ricinoleamide diethanolamine, myristamide diethanolamine, stearamide diethanolamine, oleylamide diethanolamine, tallowamide diethanolamine, lauramide monoisopropanolamine, tallowamide monoethanolamine, isostearamide diethanolamine, isostearamide diethanolamine, isostearamide monoethanolamine).

Preferably, the aqueous personal care rinse off formulation of the present invention comprises 0.1 to 30 wt % (preferably, 0.5 to 25 wt %; more preferably, 0.6 to 20 wt %; most preferably, 1 to 15 wt %), based on weight of the aqueous personal care rinse off formulation, of an alkyl polysaccharide thickener, wherein the alkyl polysaccharide thickener comprises 50 to 100 wt % (preferably, 75 to 100 wt %; more preferably, 80 to 100 wt %; still more preferably, 90 to 100 wt %; still more preferably, 95 to 100 wt %; still yet more preferably, 98 to 100 wt %; yet still more preferably, 99 to 100 wt %; most preferably, 100 wt %), based on weight of the alkyl polysaccharide thickener, of a linear alkyl polysaccharide of formula A

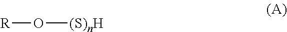

wherein R is a linear C₉₋₁₅ alkyl group (preferably, a C₉₋₁₄ alkyl group; more preferably, a C₉₋₁₃ alkyl group; most preferably, a C₁₀₋₁₂ alkyl group); wherein n is an average of 1 to 2.5 (preferably, 1.1 to 2; more preferably, 1.1 to 1.75; most preferably, 1.15 to 1.5) and wherein S is a monosaccharide selected from the group consisting of ribulose, xylulose, ribose, arabinose, xylose, lyxose and mixtures thereof (preferably, arabinose, xylose and mixtures thereof; most preferably, xylose); and wherein 70 to 100 wt % of the monosaccharide S (preferably, 80 to 100 wt %; more preferably, 85 to 100 wt %; still more preferably, 90 to 100 wt %; yet more preferably, 95 to 100 wt %; still yet more preferably, 98 to 100 wt %; most preferably, 99 to 100 wt %), based on weight of monosaccharide S in the linear alkyl polysaccharide of formula A, is xylose; with the proviso that the alkyl polysaccharide thickener comprises <15 wt % (preferably, <10 wt %; more preferably, <1 wt %; still more preferably, <0.1 wt %; yet more preferably, 0.01 wt %; still yet more preferably, 0.001 wt %; most preferably, less than detectable limit), based on weight of the alkyl polysaccharide thickener, of branched alkyl polysaccharide of formula I

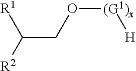

wherein R¹ is a linear or branched C₃₋₄ alkyl group; wherein R² is a linear or branched C₅₋₆ alkyl group; wherein G¹ is a monosaccharide with 4 to 6 carbon atoms and wherein x is 1.1 to 4; and with the proviso that the alkyl polysaccharide thickener comprises <1.5 wt % (preferably, <1 wt %; more preferably, <0.5 wt %; still more preferably, <0.1 wt %; yet more preferably, <0.01 wt %; still yet more preferably, <0.001 wt %; yet still more preferably, <0.0001 wt %; most preferably, less than detectable limit), based on weight of the alkyl polysaccharide thickener, of branched alkylpolysaccharide of formula II

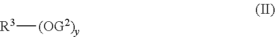

wherein R³ is an alkyl or alkenyl moiety containing 7 to 30 carbon atoms with branching at the 2 or higher carbon position on R³, wherein the branching is selected from the group consisting of methyl, ethyl, isopropyl, n-propyl, butyl, pentyl, hexyl and mixtures thereof; wherein G² is a saccharide moiety selected from the group consisting of glucose, fructose, mannose, galactose, talose, gulose, allose, altrose, idose, arabinose, xylose, lyxose, ribose and mixtures thereof and wherein y is an average of ≥2 (preferably, 2 to 10).

Preferably, the aqueous personal care rinse off formulation of the present invention comprises 0.1 to 30 wt % (preferably, 0.5 to 25 wt %; more preferably, 0.6 to 20 wt %; most preferably, 1 to 15 wt %), based on weight of the aqueous personal care rinse off formulation, of an alkyl polysaccharide thickener, wherein the alkyl polysaccharide thickener comprises 50 to 100 wt % (preferably, 75 to 100 wt %; more preferably, 80 to 100 wt %; still more preferably, 90 to 100 wt %; still more preferably, 95 to 100 wt %; still yet more preferably, 98 to 100 wt %; yet still more preferably, 99 to 100 wt %; most preferably, 100 wt %), based on weight of the alkyl polysaccharide thickener, of a linear alkyl polysaccharide of formula A

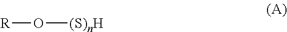

wherein R is a linear C₉₋₁₅ alkyl group (preferably, a C₉₋₁₄ alkyl group; more preferably, a C₉₋₁₃ alkyl group; most preferably, a C₁₀₋₁₂ alkyl group); wherein n is an average of 1 to 2.5 (preferably, 1.1 to 2; more preferably, 1.1 to 1.75; most preferably, 1.15 to 1.5) and wherein S is a monosaccharide selected from the group consisting of ribulose, xylulose, ribose, arabinose, xylose, lyxose and mixtures thereof (preferably, arabinose, xylose and mixtures thereof; most preferably, xylose); and wherein 70 to 100 wt % of the monosaccharide S (preferably, 80 to 100 wt %; more preferably, 85 to 100 wt %; still more preferably, 90 to 100 wt %; yet more preferably, 95 to 100 wt %; still yet more preferably, 98 to 100 wt %; most preferably, 99 to 100 wt %), based on weight of monosaccharide S in the linear alkyl polysaccharide of formula A, is xylose; with the proviso that the alkyl polysaccharide thickener comprises <15 wt % (preferably, <10 wt %; more preferably, <1 wt %; still more preferably, <0.1 wt %; yet more preferably, 0.01 wt %; still yet more preferably, 0.001 wt %; most preferably, less than detectable limit), based on weight of the alkyl polysaccharide thickener, of branched alkyl polysaccharide of formula I

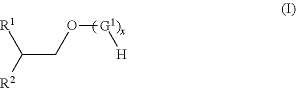

wherein R¹ is a linear or branched C₃₋₄ alkyl group; wherein R² is a linear or branched C₅₋₆ alkyl group; wherein G¹ is a monosaccharide with 4 to 6 carbon atoms and wherein x is 1.1 to 4; with the proviso that the alkyl polysaccharide thickener comprises <1.5 wt % (preferably, <1 wt %; more preferably, <0.5 wt %; still more preferably, <0.1 wt %; yet more preferably, <0.01 wt %; still yet more preferably, <0.001 wt %; yet still more preferably, <0.0001 wt %; most preferably, less than detectable limit), based on weight of the alkyl polysaccharide thickener, of branched alkylpolysaccharide of formula II

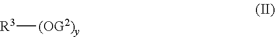

wherein R³ is an alkyl or alkenyl moiety containing 7 to 30 carbon atoms with branching at the 2 or higher carbon position on R³, wherein the branching is selected from the group consisting of methyl, ethyl, isopropyl, n-propyl, butyl, pentyl, hexyl and mixtures thereof; wherein G² is a saccharide moiety selected from the group consisting of glucose, fructose, mannose, galactose, talose, gulose, allose, altrose, idose, arabinose, xylose, lyxose, ribose and mixtures thereof and wherein y is an average of ≥2 (preferably, 2 to 10); and with any one or more of the following additional provisos (i)-(x), namely: (i) with the proviso that the alkyl polysaccharide thickener comprises <1 wt % (preferably, <0.5 wt %; more preferably, <0.1 wt %; still more preferably, <0.01 wt %; yet more preferably, <0.001 wt %; still yet more preferably, <0.0001 wt %; most preferably, less than detectable limit), based on weight of the alkyl polysaccharide thickener, of branched alkyl polysaccharide of formula III

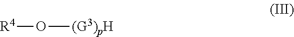

wherein R⁴ is a branched C₈₋₁₅ alkyl group; wherein G³ is a monosaccharide with 5 or 6 carbon atoms and wherein p is an average of 1 to 10; (ii) with the proviso that the alkyl polysaccharide thickener comprises <10 wt % (preferably, <1 wt %; more preferably, <0.1 wt %; still more preferably, <0.01 wt %; yet more preferably, <0.001 wt %; still yet more preferably, <0.0001 wt %; yet still more preferably, <0.00001 wt %; most preferably, less than detectable limit), based on weight of the alkyl polysaccharide thickener, of branched alkyl polysaccharide of formula IV

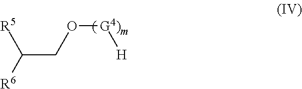

wherein R⁵ is —(CH₂)ᵣCH(CH₃)₂; wherein R⁶ is —(CH₂)ᵣ₊₂CH(CH₃)₂; G⁴ is a monosaccharide with 4 to 6 carbon atoms; wherein m is 1.1 to 4 and wherein r is 0 to 3; (iii) with the proviso that the alkyl polysaccharide thickener comprises <1.5 wt % (preferably, <1 wt %; more preferably, <0.1 wt %; still more preferably, <0.01 wt %; yet more preferably, <0.001 wt %; still yet more preferably, <0.0001 wt %; yet still more preferably, <0.00001 wt %; most preferably, less than detectable limit), based on weight of the alkyl polysaccharide thickener, of branched alkyl polysaccharide of formula V

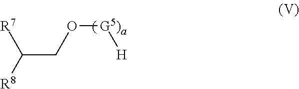

wherein R⁷ is —(CH₂)_{b}CH(CH₃)₂; wherein R⁸ is —CH₂—CH(CH₃)(CH₂)_{b+1}CH₃; G⁵ is a monosaccharide with 4 to 6 carbon atoms; wherein a is 1.1 to 4 and wherein b is 0 to 3; (iv) with the proviso that the alkyl polysaccharide thickener comprises <0.5 wt % (preferably, <0.1 wt %; more preferably, <0.05 wt %; still more preferably, <0.01 wt %; yet more preferably, <0.001 wt %; still yet more preferably, <0.0001 wt %; yet still more preferably, <0.00001 wt %; most preferably, less than detectable limit), based on weight of the alkyl polysaccharide thickener, of branched alkyl polysaccharide of formula VI

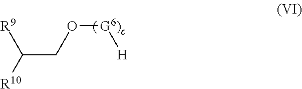

wherein R⁹ is —(CH₂)_{d+1}CH₃; wherein R¹⁰ is —(CH₂)_{d+3}CH₃; wherein G⁶ is monosaccharide with 4 to 6 carbon atoms; wherein d is 0 to 3 and wherein c is 1.1 to 4; (v) with the proviso that the alkyl polysaccharide thickener comprises <0.05 wt % (preferably, <0.01 wt %; more preferably, <0.005 wt %; still more preferably, <0.001 wt %; yet more preferably, <0.0001 wt %; still yet more preferably, <0.00001 wt %; most preferably, less than detectable limit), based on weight of the alkyl polysaccharide thickener, of compound of formula VII

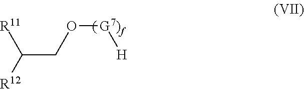

wherein R¹¹ is a hydrogen or a linear or branched C₁₋₄ alkyl group; wherein R¹² is a —CH₂CH₂R¹¹ group; wherein G⁷ is glucose or xylose, wherein an amount of glucose ranges from 70 to 95 mol % and an amount of xylose ranges from 5 to 30 mol % and wherein f is 1.1 to 4; (vi) with the proviso that the alkyl polysaccharide thickener comprises <5 wt % (preferably, <1 wt %; more preferably, <0.5 wt %; still more preferably, <0.1 wt %; yet more preferably, <0.01 wt %; still yet more preferably, <0.001 wt %; yet still more preferably, <0.0001 wt %; most preferably, less than detectable limit), based on weight of the alkyl polysaccharide thickener, of branched alkyl polysaccharide of formula VIII

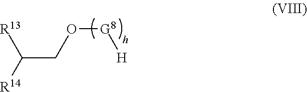

wherein R¹³ is —(CH₂)ₖCH₃; wherein R¹⁴ is —(CH₂)ₖ₊₂CH₃; wherein G⁸ is a monosaccharide with 4 to 6 carbon atoms; wherein h is 1.1 to 10 and wherein k is 1 to 4; (vii) with the proviso that the alkyl polysaccharide thickener comprises <1 wt % (preferably, <0.5 wt %; more preferably, <0.1 wt %; still more preferably, <0.05 wt %; yet more preferably, <0.01 wt %; still yet more preferably, <0.001 wt %; yet still more preferably, <0.0001 wt %; most preferably, less than detectable limit), based on weight of the alkyl polysaccharide thickener, of branched alkyl polysaccharide of formula IX

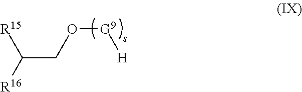

wherein R¹⁵ is —(CH₂)ₜCH₃; wherein R¹⁶ is —(CH₂)ₜ₊₁CH(CH₃)₂; wherein G⁹ is a monosaccharide with 4 to 6 carbon atoms; wherein s is 1.1 to 10 and wherein t is 1 to 4; (viii) with the proviso that the alkyl polysaccharide thickener comprises <0.2 wt % (preferably, <0.1 wt %; more preferably, <0.05 wt %; still more preferably, <0.01 wt %; yet more preferably, <0.001 wt %; still yet more preferably, <0.0001 wt %; yet still more preferably, <0.00001 wt %; most preferably, less than detectable limit), based on weight of the alkyl polysaccharide thickener, of branched alkyl polysaccharide of formula X

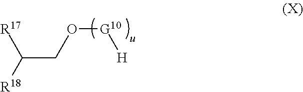

wherein R¹⁷ is —(CH₂)ᵥ₊₁CH(CH₃)₂; wherein R¹⁸ is —(CH₂)ᵥ₋₁CH(CH₃)₂; wherein G¹⁰ is a monosaccharide with 4 to 6 carbon atoms; wherein u is 1.1 to 10 and wherein v is 1 to 4; (ix) with the proviso that when the alkyl polysaccharide thickener comprises both fatty acid ester C₁₂₋₂₃ alkyl-(O)O— moieties and fatty acid ester C₆₋₁₀ alkyl-(O)O— moieties, the mole ratio of the C₁₂₋₂₃ alkyl-(O)O— moieties to the C₆₋₁₀ alkyl-(O)O— moieties is >1.1:1 (preferably, >1.2:1; more preferably, >1.3:1; still more preferably, >1.5:1; most preferably, >2:1); and/or (x) with the proviso that the alkyl polysaccharide thickener comprises <0.1 wt % (preferably, <0.05 wt %; more preferably, <0.01 wt %; still more preferably, <0.001 wt %; yet more preferably, <0.0001 wt %; still yet more preferably, <0.00001 wt %; most preferably, less than detectable limit), based on weight of the alkyl polysaccharide thickener, of linear alkyl polysaccharide of formula XIII

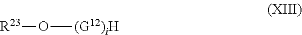

wherein R²³ is a linear C₁₋₈ alkyl group; wherein G¹² is a monosaccharide with 5 or 6 carbon atoms and wherein i is an average of 1 to 10.

Preferably, the aqueous personal care rinse off formulation of the present invention, further comprises at least one ingredient selected from the group consisting of an absorbent; an aesthetic enhancer (e.g., starch); an alpha hydroxy acid; an antiaging agent; an antidandruff agent; an antifungal; an antimicrobial agent; an antioxidant (e.g., butylated hydroxytoluene); an antiseptic; an antistatic agent; a bioactive agent; a bleaching agent; a chelating agent; a colorant; a conditioning agent (e.g., silicone, polyquaternium, cationic guar); a dye; an emollient; an emulsifying agent; a film former (e.g., water proofing agent); a fixative polymer; a foaming agent; a fragrance; a hard particle; a humectant (e.g., glycerin, sorbitol, monoglycerides, lecithins, glycolipids, fatty alcohols, fatty acids, polysaccharides, sorbitan esters, polysorbates (e.g., Polysorbate 20, Polysorbate 40, Polysorbate 60, and Polysorbate 80), diols (e.g., propylene glycol), diol analogs, triols, triol analogs, cationic polymeric polyols); a lubricating agent; an opacifier, a pearlizing agent; a penetrant; a pH adjusting agent; a pigment; a plant extract; a preservative (e.g., benzoic acid, sorbic acid, phenoxyethanol); a protein/amino acid; a rheology modifier; a salt (e.g., NaCl); a sensory modifier; a slip agent; a soap; a soft particle; a sunscreen additive; a suspending agent; a UV light inhibitor, a vitamin and mixtures thereof.

Preferably, the aqueous personal care rinse off formulation of the present invention, further comprises 0 to 10 wt % (preferably, 0 to 3 wt %), based on weight of the aqueous personal care rinse off formulation, of a salt (e.g., NaCl).

Preferably, the aqueous personal care rinse off formulation of the present invention, further comprises an antimicrobial agent. More preferably, the aqueous personal care rinse off formulation of the present invention, further comprises an antimicrobial agent; wherein the antimicrobial agent is selected from the group consisting of phenoxyethanol, benzoic acid, benzyl alcohol, sodium benzoate, DMDM hydantoin, 2-ethylhexyl glyceryl ether and isothiazolinone (e.g., methylchloroisothiazolinone, methylisothiazolinone). Still more preferably, the aqueous personal care rinse off formulation of the present invention, further comprises an antimicrobial agent; wherein the antimicrobial agent is an isothiazolinone (more preferably, wherein the antimicrobial is selected from the group consisting of methylisothiazolinone, methylchloroisothiazolinone and mixtures thereof; most preferably, wherein the biocide is methylisothiazolinone).

Preferably, the aqueous personal care rinse off formulation of the present invention, further comprises a pH adjusting agent. More preferably, the aqueous personal care rinse off formulation of the present invention, further comprises a pH adjusting agent. Most preferably, the aqueous personal care rinse off formulation of the present invention, further comprises a pH adjusting agent; wherein the aqueous personal care rinse off formulation has a pH of 4.5 to 9 (preferably, 5 to 8; most preferably, 5 to 7).

Preferably, the pH adjusting agent is selected from the group consisting of at least one of citric acid, lactic acid, hydrochloric acid, aminoethyl propanediol, triethanolamine, monoethanolamine, sodium hydroxide, potassium hydroxide, amino-2-methyl-1-propanol. More preferably, the pH adjusting agent is selected from the group consisting of at least one of citric acid, lactic acid, sodium hydroxide, potassium hydroxide, triethanolamine, amino-2-methyl-1-propanol. Still more preferably, the pH adjusting agent includes is triethanolamine. Most preferably, the pH adjusting agent is triethanolamine.

Preferably, the aqueous personal care rinse off formulation has a viscosity ≥2,000 cP at 21° C. (preferably, 2,000 to 50,000 cP at 21° C.; more preferably, 2,500 to 30,000 cP at 21° C.; still more preferably, 3,000 to 25,000 cP at 21° C.; yet more preferably, 4,000 to 20,000 cP at 21° C.; most preferably, 5,000 to 15,000 cP at 21° C.) (preferably, measured after 24 hours in a temperature-controlled room (21±3° C.), using a Brookfield Viscosimeter Model DV-II+ at 30 RPM, with a RV spindle 31. The viscosity value can be taken after a stabilization time of 1 min).

Preferably, the aqueous personal care rinse off formulation comprises <0.05 wt % (preferably, <0.01 wt %; more preferably, <0.005 wt %; still more preferably, <0.001 wt %; yet more preferably, <0.0005 wt %; still yet more preferably, <0.0001 wt %; yet still more preferably, <0.00001 wt %; most preferably, less than detectable limit), based on weight of the aqueous personal care rinse off formulation, of branched alkyl polysaccharide of formula IV

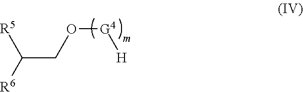

wherein R⁵ is —(CH₂)ᵣCH(CH₃)₂; wherein R⁶ is —(CH₂)ᵣ₊₂CH(CH₃)₂; G⁴ is a monosaccharide with 4 to 6 carbon atoms; wherein m is 1.1 to 4 and wherein r is 0 to 3.

Preferably, the aqueous personal care rinse off formulation comprises <0.1 wt % (preferably, <0.01 wt %; more preferably, <0.001 wt %; still more preferably, <0.0001 wt %; yet more preferably, <0.00001 wt %; most preferably, less than detectable limit), based on weight of the aqueous personal care rinse off formulation, of branched alkyl polysaccharide according to formula XI

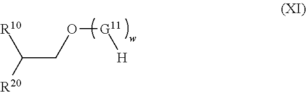

wherein R¹⁹ is a C₂₋₄ alkyl group; wherein R²⁰ is a C₄₋₇ alkyl group; wherein the sum of the carbon atoms in R¹⁹ and R²⁰ is 7 to 11; wherein G¹¹ is a monosaccharide (i.e., glucose, mannose, galactose, talose, allose, altrose, idose, arabinose, xylose, ribose, lyxose and mixtures thereof) and wherein w is 1 to 4.

Preferably, the aqueous personal care rinse off formulation comprises <0.05 wt % (preferably, <0.01; more preferably, <0.005; still more preferably, <0.001; yet more preferably, <0.0005; still yet more preferably, <0.0001 wt %; most preferably, less than detectable limit), based on weight of the aqueous personal care rinse off formulation, of a fatty acid alkyl ester alkoxylate of formula XII

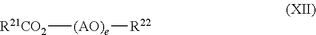

wherein R²¹ is a linear or branched, saturated or unsaturated, alkyl group containing 5 to 21 carbon atoms; wherein AO is a C₂₋₄ alkylene oxide group; wherein e is 1 to 30 and wherein R²² is a linear or branched alkyl group containing 1 to 6 carbon atoms.

Preferably, the aqueous personal care rinse off formulation comprises <0.01 wt % (preferably, <0.005 wt %; more preferably, <0.001 wt %; still more preferably, <0.0001 wt %; yet more preferably, <0.00001 wt %; most preferably, less than detectable limit), based on weight of the aqueous personal care rinse off formulation, of alkyl polyglucoside (e.g., a C₁₋₃₀ alkyl polyglucoside).

Preferably, the aqueous personal care rinse off formulation comprises <0.1 wt % (preferably, <0.05 wt %; more preferably, <0.01 wt %; still more preferably, <0.001 wt %; yet more preferably, <0.0001 wt %; most preferably, less than detectable limit), based on weight of the aqueous personal care rinse off formulation, of C₆₋₂₄ alkyl polyglucoside (e.g., polyglucoside).

Preferably, the aqueous personal care rinse off formulation comprises <0.01 wt % (preferably, <0.005 wt %; more preferably, <0.001 wt %; still more preferably, <0.0001 wt %; yet more preferably, <0.00001 wt %; most preferably, less than detectable limit), based on weight of the aqueous personal care rinse off formulation, of alkyl polysaccharide comprising fatty acid ester C₁₂₋₂₃ alkyl-(O)O— moieties and/or fatty acid ester C₆₋₁₀ alkyl-(O)O— moieties.

Preferably, the method of cleaning at least one of mammalian skin and hair (preferably, at least one of human skin and hair) of the present invention, comprises: applying an aqueous personal care rinse off formulation of the present invention to the skin or hair of a mammal (preferably, skin or hair of a human); and rinsing the aqueous personal care rinse off formulation from the skin or hair with a rinse water.

Some embodiments of the present invention will now be described in detail in the following Examples.

### Syntheses S1-S10: Alkyl Polysaccharides

A 500 mL round-bottomed reactor was placed in an aluminum heating block and fitted with a 25 mL Dean-Stark trap (containing 15 mL hexanes) and a magnetic stir bar. To the reactor was added sugar (A g, 200 mmol) and n-butanol (B mL, 1.2 mol, 6 equiv) as noted in TABLE 1. The reactor contents were then stirred vigorously under a nitrogen blanket (bubbler). To the stirring reactor contents was added para-toluenesulfonic acid monohydrate (C mg, 2.00 mmol, 1 mol %)(noted in TABLE 1) and the aluminum heating block set at temperature T¹ (noted in TABLE 1) and maintained at that temperature for 2 hours. The reactor contents were then allowed to cool to ambient temperature with continued stirring. Then alcohol (D, 600 mmol, 3 equiv) was added to the reactor contents. The type and amount of the alcohol added are reported in TABLE 1. The Dean-Stark trap was then replated with a still head and a condenser flask. The reactor contents were then vacuum distilled with the aluminum heating block set at temperature T² (noted in TABLE 1) and the condenser flask set in an ice-cooled bath. The vacuum was periodically adjusted to a flow rate of 1-2 drops/second. The vacuum was maintained for 2 hours. The distillation was then stopped. The distillation apparatus was placed back under a nitrogen atmosphere and the stirred contents of the reactor were allowed to cool to ambient temperature. A solution of sodium hydroxide (120 μL, 50 wt % in water, 2.2 mmol) was added to the stirring reactor contents. After vigorous stirring, the pH was measured (pH paper) to ensure the reactor contents were basic. A 30 mL sample of the reactor contents was diluted with hexanes (30 mL) and purified via automated column chromatography (silica, 220 g). The alcohol eluted at 20/80 ethyl acetate/hexanes. The solvent mixture was then ramped over 2 column volumes to 100% ethyl acetate. The solvent system was then changed to ethyl acetate-methanol, and the solvent ratio was ramped over 6 column volumes to neat methanol—during which time the product eluted as a mixture of mono- and polysaccharides. The solvent was removed under reduced pressure to give the final product.

TABLE 1

| | Sugar | | B | C | Alcohol | | | Temp. (° C.) | |
|---|---|---|---|---|---|---|---|---|---|
| Ex. | Type | A (g) | (mL) | (mg) | Type | D | | T¹ | T² |
| S1 | glucose | 25 | 65 | 268 | C₁₀ | 106 | mL | 125 | 130 |
| S2 | glucose | 15 | 45 | 160 | C₁₁ | 70 | mL | 125 | 125 |
| S3 | glucose | 15 | 45 | 160 | C₁₂ | 69 | g | 125 | 130 |
| S4 | glucose | 15 | 40 | 162 | C₁₄ | 73 | g | 125 | 130 |
| S5 | xylose | 30 | 110 | 760 | C₈ | 94 | mL | 135 | 135 |
| S6 | xylose | 30 | 110 | 380 | C₉ | 104 | mL | 138 | 135 |
| S7 | xylose | 30 | 110 | 380 | C₁₀ | 114 | mL | 135 | 135 |
| S8 | xylose | 30 | 110 | 380 | C₁₁ | 124 | mL | 137 | 135 |
| S9 | xylose | 30 | 110 | 380 | C₁₂ | 112 | g | 136 | 135 |
| S10 | xylose | 30 | 110 | 380 | C₁₄ | 129 | g | 135 | 135 |

### Comparative Examples CF1-CF5 and Examples F1-F5: Aqueous Formulations

Aqueous surfactant formulations were prepared having composition according to Comparative Examples CF1-CF5 and Examples F1-F5 as noted in TABLE 2.

TABLE 2

| | Example (wt %) | | | | | | | | | |
|---|---|---|---|---|---|---|---|---|---|---|
| Ingredient | CF1 | CF2 | CF3 | CF4 | CF5 | F1 | F2 | F3 | F4 | F5 |
| Cocamidopropyl | 8.4 | 7.5 | 7.5 | 7.5 | 7.5 | 7.5 | 7.5 | 7.5 | 7.5 | 7.5 |
| betaine¹ | | | | | | | | | | |
| Sodium laureth | 4.1 | 3.7 | 3.7 | 3.7 | 3.7 | 3.7 | 3.7 | 3.7 | 3.7 | 3.7 |
| sulfate² | | | | | | | | | | |
| Sodium chloride | 2.0 | 2.0 | 2.0 | 2.0 | 2.0 | 2.0 | 2.0 | 2.0 | 2.0 | 2.0 |
| (NaCl) | | | | | | | | | | |
| Synthesis S1 | — | 1.25 | — | — | — | — | — | — | — | — |
| Synthesis S2 | — | — | 1.25 | — | — | — | — | — | — | — |
| Synthesis S3 | — | — | — | 1.25 | — | — | — | — | — | — |
| Synthesis S4 | — | — | — | — | 1.25 | — | — | — | — | — |
| Synthesis S6 | — | — | — | — | — | 1.25 | — | — | — | — |
| Synthesis S7 | — | — | — | — | — | — | 1.25 | — | — | — |
| Synthesis S8 | — | — | — | — | — | — | — | 1.25 | — | — |
| Synthesis S9 | — | — | — | — | — | — | — | — | 1.25 | — |
| Synthesis S10 | — | — | — | — | — | — | — | — | — | 1.25 |

| | |
|---|---|
| Deionized water | q.s. 100 |

¹Stepan Amphosol CA from Stepan Company
²STEOL CS-330 from Stepan Company

### Thickening Performance

The thickening performance of the aqueous surfactant thickening performance of the alkyl polysaccharides of Syntheses S1-S4 and S6-S10 was assessed by measuring the viscosity of the aqueous surfactant formulations prepared according to Comparative Examples CF1-CF5 and Examples F1-F5. The viscosities reported in TABLE 3 were measured by using the Total Aspiration and Dispense Monitoring (TADM) function on a Hamilton MicroLab Star liquid handling robot at 21° C. The Hamilton aspirates and dispenses the samples at a set speed, and monitors the pressure changes (Δp). Calibration curves are generated using known viscosity standards. And viscosity is calculated by comparing the pressure curves of the samples against the pressure curves of viscosity standards. The data collected by TADM method was the Δp value in pascals (Pa). The viscosity was calculated from the measured Δp according to the formula: Viscosity=0.3461×Δp−115.44.

TABLE 3

| Aqueous surfactant | Alkyl | Viscosity |
|---|---|---|
| formulation | polysaccharide | (cP) |
| Comp. Example CF1 | None | 481 |
| Comp. Example CF2 | Synthesis S1 | 438 |
| Comp. Example CF3 | Synthesis S2 | 1,322 |
| Comp. Example CF4 | Synthesis S3 | 1,420 |
| Comp. Example CF5 | Synthesis S4 | 1,620 |
| Example F1 | Synthesis S6 | 2,535 |
| Example F2 | Synthesis S7 | 4,161 |
| Example F3 | Synthesis S8 | 4,836 |
| Example F4 | Synthesis S9 | 7,320 |
| Example F5 | Synthesis S10 | 3,810 |

### Comparative Examples CF6-CF8 and Example F6: Aqueous Surfactant Formulations

Aqueous surfactant formulations were prepared having composition according to Comparative Examples CF6-CF8 and Example F6 as noted in TABLE 4.

TABLE 4

| | Example (wt %) | | | |
|---|---|---|---|---|
| Ingredient | CF6 | CF7 | CF8 | F6 |
| Cocamidopropyl betaine¹ | 7.5 | 7.5 | 7.5 | 7.5 |
| Sodium laureth sulfate² | 3.75 | 3.75 | 3.75 | 3.75 |
| Sodium chloride (NaCl) | 2.0 | 2.0 | 2.0 | 2.0 |
| Synthesis S5 | 1.25 | 0.625 | 0.25 | — |
| Synthesis S7 | — | 0.310 | 0.50 | 0.625 |
| Synthesis S9 | — | 0.310 | 0.50 | 0.625 |

| | |
|---|---|
| Deionized water | q.s. 100 |

¹Stepan Amphosol CA from Stepan Company
²STEOL CS-330 from Stepan Company

### Thickening Performance

The influence of short alkyl chain (C₈ alkyl) alkyl polysaccharide (Synthesis S5) on blends of longer chain (C₁₀/C₁₂ alkyl) alkyl polysaccharides (Syntheses S7 and S9) was assessed by measuring the viscosity of the aqueous surfactant formulations prepared according to Comparative Examples CF6-CF8 and Example F6. The viscosities reported in TABLE 5 were measured by using the Total Aspiration and Dispense Monitoring (TADM) function on a Hamilton MicroLab Star liquid handling robot at 21° C. The Hamilton aspirates and dispenses the samples at a set speed, and monitors the pressure changes (Δp). Calibration curves are generated using known viscosity standards. And viscosity is calculated by comparing the pressure curves of the samples against the pressure curves of viscosity standards. The data collected by TADM method was the Δp value in pascals (Pa). The viscosity was calculated from the measured Δp according to the formula: Viscosity=0.3461×Δp−115.44.

TABLE 5

| Aqueous surfactant | | Viscosity |
|---|---|---|
| formulation | Alkyl polysaccharide | (cP) |
| Comp. Example CF6 | None | 1,285 |
| Comp. Example CF7 | Synthesis S5 | 2,652 |
| Comp. Example CF8 | Syntheses S5, S7 and S9 | 4,347 |
| Example F6 | Syntheses S7 and S9 | 5,776 |

### Comparative Examples CF9-CF11 and Examples F7-8: Aqueous Formulations

Aqueous surfactant formulations were prepared having composition according to Comparative Examples CF9-CF11 and Examples F7-8 as noted in TABLE 6.

TABLE 6

| | Example (wt %) | | | | |
|---|---|---|---|---|---|
| Ingredient | CF9 | CF10 | CF11 | F7 | F8 |
| Cocami- | 4 | 3.6 | 3.6 | 3.6 | 3.6 |
| dopropyl | | | | | |
| betaine¹ | | | | | |
| Sodium | 2 | 1.8 | — | 1.8 | — |
| laureth | | | | | |
| sulfate² | | | | | |
| Sodiul lauryl | — | — | 1.8 | — | 1.8 |
| sulfate³ | | | | | |
| Synthesis S1 | — | 0.6 | 0.6 | — | — |
| Synthesis S7 | — | — | — | 0.6 | 0.6 |
| Deionized | q.s. 100 | | | | |
| water | | | | | |

¹Stepan Amphosol CA from Stepan Company
²STEOL CS-170 UB from Stepan Company
³Stepanol WA EXTRA from Stepan Company

### Thickening Performance

The superior thickening performance of C₁₀ alkyl polyxyloside to that of C₁₀ alkyl polyglucoside was demonstrated by comparing the viscosity measured for the aqueous surfactant formulations prepared with C₁₀ alkyl polyglucoside according to Comparative Examples CF9-CF11 with that of the aqueous surfactant formulations prepared with C₁₀ alkyl polyxyloside according to Examples F7-8. The viscosities reported in TABLE 7 were measured by using the Total Aspiration and Dispense Monitoring (TADM) function on a Hamilton MicroLab Star liquid handling robot at 21° C. The Hamilton aspirates and dispenses the samples at a set speed, and monitors the pressure changes (Δp). Calibration curves are generated using known viscosity standards. And viscosity is calculated by comparing the pressure curves of the samples against the pressure curves of viscosity standards. The data collected by TADM method was the Δp value in pascals (Pa). The viscosity was calculated from the measured Δp according to the formula: Viscosity=0.3461×Δp−115.44.

TABLE 7

| Aqueous surfactant | Alkyl | Viscosity |
|---|---|---|
| formulation | polysaccharide | (cP) |
| Comp. Example CF9 | None | 1,939 |
| Comp. Example CF10 | Synthesis S1 | 1,650 |
| Comp. Example CF11 | Synthesis S1 | 2,167 |
| Example F7 | Synthesis S10 | 2,833 |
| Example F8 | Synthesis S10 | 9,738 |

We claim:

1. An aqueous personal care rinse off formulation comprising:
a dermatologically acceptable aqueous vehicle;
a dermatologically acceptable cleaning surfactant, wherein the dermatologically acceptable cleaning surfactant comprises an anionic surfactant; and
an alkyl polysaccharide thickener, wherein the alkyl polysaccharide thickener comprises 50 to 100 wt %, based on weight of the alkyl polysaccharide thickener, of a linear alkyl polysaccharide of formula A 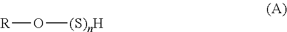
wherein R is a linear C₉₋₁₅ alkyl group; wherein n is an average of 1 to 2.5 and wherein S is a monosaccharide selected from the group consisting of ribulose, xylulose, ribose, arabinose, xylose, lyxose and mixtures thereof; and wherein 70 to 100 wt % of the monosaccharide S, based on weight of monosaccharide S in the linear alkyl polysaccharide of formula A, is xylose;
with the proviso that the alkyl polysaccharide thickener comprises <15 wt %, based on weight of the alkyl polysaccharide thickener, of branched alkyl polysaccharide of formula I 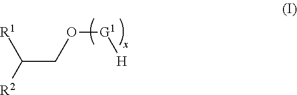
wherein R¹ is a linear or branched C₃₋₄ alkyl group; wherein R² is a linear or branched C₅₋₆ alkyl group; wherein G¹ is a monosaccharide with 4 to 6 carbon atoms and wherein x is 1.1 to 4; and
with the proviso that the alkyl polysaccharide thickener comprises <1.5 wt %, based on weight of the alkyl polysaccharide thickener, of branched alkylpolysaccharide of formula II 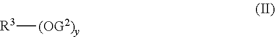
wherein R³ is an alkyl or alkenyl moiety containing 7 to 30 carbon atoms with branching at the 2 or higher carbon position on R³, wherein the branching is selected from the group consisting of methyl, ethyl, isopropyl, n-propyl, butyl, pentyl, hexyl and mixtures thereof; wherein G² is a saccharide moiety selected from the group consisting of glucose, fructose, mannose, galactose, talose, gulose, allose, altrose, idose, arabinose, xylose, lyxose, ribose and mixtures thereof and wherein y is an average of ≥2.

2. The aqueous personal care rinse off formulation of claim 1, wherein the anionic surfactant is selected from the group consisting of glycinates, taurates, glutamates, sarcosinates, isethionates, sulfoacetates, alaninates, sulfates, sulfonates, succinates, carboxylates and mixtures thereof.

3. The aqueous personal care rinse off formulation of claim 2, wherein the dermatologically acceptable cleaning surfactant further comprises a amphoteric surfactant.

4. The aqueous personal care rinse off formulation of claim 3, wherein the amphoteric surfactant is selected from the group consisting of betaines, amphoacetates, sultaines and mixtures thereof.

5. The aqueous personal care rinse off formulation of claim 4, wherein the anionic surfactant comprises sodium laureth sulfate and the amphoteric surfactant comprises cocamidopropyl betaine.

6. The aqueous personal care rinse off formulation of claim 5, wherein R is a linear C₁₀₋₁₂ alkyl group.

7. The aqueous personal care rinse off formulation of claim 1, wherein the aqueous personal care rinse off formulation has a viscosity ≥1,500 cP at 21° C.

8. The aqueous personal care rinse off formulation of claim 7, further comprising an ingredient selected from the group consisting of an absorbent, an aesthetic enhancer, an alpha hydroxy acid, an antiaging agent, an antidandruff agent, an antifungal, an antimicrobial agent, an antioxidant, an antiseptic, an antistatic agent, a bioactive agent, a bleaching agent, a chelating agent, a colorant, a conditioning agent, a dye, an emollient, an emulsifying agent, a film former, a fixative polymer, a foaming agent, a fragrance, a hard particle, a humectant, a lubricating agent, an opacifier, a pearlizing agent, a penetrant, a pH adjusting agent, a pigment, a plant extract, a preservative, a protein/amino acid, a rheology modifier, a salt, a sensory modifier, a slip agent, a soft particle, a sunscreen additive, a suspending agent, a UV light inhibitor, a vitamin and mixtures thereof.

9. The aqueous personal care rinse off formulation of claim 8, wherein the aqueous personal care rinse off formulation is selected from the group consisting of a shampoo, a conditioning shampoo, a body wash formulation, an exfoliating body wash formulation, a facial wash formulation, an exfoliating facial wash formulation, a liquid hand soap formulation, a mild cleansing formulation.

10. A method of cleaning at least one of mammalian skin and hair, comprising:
applying an aqueous personal care rinse off formulation according to claim 1 to the skin or hair of a mammal; and
rinsing the aqueous personal care rinse off formulation from the skin or hair with a rinse water.
